(19) United States
(12) Patent Application Publication
(10) Pub. No.: US 2024/0415954 A1
(43) Pub. Date: Dec. 19, 2024

(54) VACCINE PREPARATION

(71) Applicant: HCEMM Nonprofit Kft., Szeged (HU)

(72) Inventor: Stefan GRABUSCHNIG, Graz (AT)

(73) Assignee: HCEMM Nonprofit Kft., Szeged (HU)

(21) Appl. No.: 18/718,892

(22) PCT Filed: Dec. 1, 2022
(86) PCT No.: PCT/EP2022/084011

§ 371 Date: Jun. 12, 2024

(30) Foreign Application Priority Data
Dec. 16, 2021 (AT) A 51010/2021

(51) Int. Cl. A61K 39/215; A61K 31/137; A61K 31/35; A61K 31/4045; A61K 38/22; A61K 39/00

(52) U.S. Cl. CPC A61K 39/215; A61K 31/137; A61K 31/35; A61K 31/4045; A61K 38/22; A61K 2039/53

(57) ABSTRACT

A vaccine preparation to be administered intramuscularly without or at least minor adverse effects comprises in addition to a mRNA-vaccine at least one vasoconstrictive agent, such as epinephrine, levonorderfrine and norepinephrine.

The invention concerns a vaccine preparation, which contains at least one vaccine.

Since the first mRNA-vaccines [Chaudhary, N., Weissman, D. & Whitehead, K. A. mRNA vaccines for infectious diseases: principles, delivery and clinical translation. Nat. Rev. Drug Discov. 20, 817-838 (2021)] against COVID-19 were introduced by Pfizer-BioNTech [Benjamini, O. et al. Safety and efficacy of the BNT162b mRNA COVID-19 vaccine in patients with chronic lymphocytic leukemia. Haematologica 107, 625-634 (2021)] and by Moderna [Baden, L. R. et al. Efficacy and Safety of the mRNA-1273 SARS-CoV-2 Vaccine. N. Engl. J. Med. 384, 403-416 (2021)], this new vaccine technology has received broad public attention. Although these vaccines showed a high rate of protection and a good tolerability, also rare complications, such as cases of myocarditis, were reported in some instances [Mevorach, D. et al. Myocarditis after BNT162b2 mRNA Vaccine against Covid-19 in Israel. N. Engl. J. Med. 385, 2140-2149 (2021) and Koizumi, T. et al. Myocarditis after COVID-19 mRNA vaccines. QJM Int. J. Med. 114, 741-743 (2021)]. Experiments performed on mice indicated, that the bloodstream entry of mRNA-vaccines relates to a substantially increased risk of myocardial inflammations [Li, C. et al. Intravenous Injection of Coronavirus Disease 2019 (COVID-19) mRNA Vaccine Can Induce Acute Myopericarditis in Mouse Model. Clin. Infect. Dis. 74, 1933-1950 (2022)]. Even though the application of mRNA-vaccines occurs via an intramuscular injection, injury of blood vessels is always possible during injection, which may facilitate bloodstream entry [Li, C. et al. Intravenous Injection of Coronavirus Disease 2019 (COVID-19) mRNA Vaccine Can Induce Acute Myopericarditis in Mouse Model. Clin. Infect. Dis. 74, 1933-1950 (2022) and Arin, Kim. Is injection technique responsible for vaccine side effects? Korean Her. (2021)].

It is an established procedure to combine local anesthetics with a vasoconstrictive agent, such as adrenalin, before injection [Sisk, A. L. Vasoconstrictors in local anesthesia for dentistry. Anesth. Prog. 39, 187-193 (1992)]. This leads to a significantly reduced migration of the anesthetic drug through the tissue, which comes with the benefit of an enhanced and prolonged anesthetic effect [Sisk, A. L. Vasoconstrictors in local anesthesia for dentistry. Anesth. Prog. 39, 187-193 (1992) and Åberg, G., Dhunér, K.-G. & Sydnes, G. Studies on the Duration of Local Anaesthesia: Structure/Activity Relationships in a Series of Homologous Local Anaesthetics. Acta Pharmacol. Toxicol. (Copenh.) 41, 432-443 (2009)].

Addition of vasoconstrictors to local anesthetics occurs in the following exemplary concentrations [Sisk, A. L. Vasoconstrictors in local anesthesia for dentistry. Anesth. Prog. 39, 187-193 (1992)]:
Adrenalin/Epinephrine:
   1: 50,000 (0.02 mg/mL)
   1: 100,000 (0.01 mg/mL)
   1: 200,000 (0.005 mg/mL)
Levonordefrin:
   1: 20,000 (0,05 mg/mL)
Norepinephrine:
   1: 30,000 (0.033 mg/mL)

Local anesthetics, contain e.g., 20-500 mg of the drug Lidocaine [Fachinformation Lidocain. https://s3.eu-central-1.amazonaws.com/prod-cerebro-ifap/media_all/78264.pdf], which are applied in volumes of 0.5-1.5 mL via injection. They are combined with e.g., epinephrine in concentrations ranging from 1:200,000 to 1:100,000. Thus, this vasoconstrictive agent is applied in doses between 0.0025 and 0,015 mg.

mRNA-Vaccines:

The CoViD-19 vaccines of Moderna (Spikevax PatNr: U.S. Ser. No. 10/933,127) and Pfizer/Biontech (Comirnaty PatNr: GB2594364A) are used for the practical examples provided in the context of this document. The individual compositions of these vaccines are not described in the respective patents. These essentially only list potential ingredients. Instead, the complete lists of ingredients can be found within the respective datasheets [Product Information Comirnaty. https://www.ema.europa.eu/en/documents/product-information/comirnaty-epar-product-information en.pdf and Product Information Spikevax. https://www.ema.europa.eu/en/documents/product-information/spikevax-previously-covid-19-vaccine-moderna-epar-product-information en.pdf], which are downloadable from the homepage of the European Medical Agency (EMA). These are specified as follows:

### Comirnaty:

The vaccine is supplied via multidose vials. The content of a multidose vial (0.45 mL) is designated for 6 vaccine doses of 0.3 mL each and thus has to be diluted before usage.

A single dose (0.3 mL) contains 30 micrograms of tozinameran. Tozinameran is a single-stranded messenger-RNA (mRNA) with a 5′-cap structure embedded in lipid-nanoparticles.

The other ingredients are the following:
((4-hydroxybutyl)azanediyl)bis(hexane-6,1-diyl)bis(2-hexyldecanoate) (ALC-0315)
2-[(polyethylene glycol)-2000]-N,N-ditetradecylacetamide (ALC-0159)
1,2-Distearoyl-sn-glycero-3-phosphocholine (DSPC)
Cholesterol
Potassium chloride
Potassium dihydrogen phosphate
Sodium chloride
Disodium phosphate dihydrate
Sucrose
Water for injections
Sodium hydroxide (for pH adjustment)
Hydrochloric acid (for pH adjustment)

### Spikevax:

The vaccine is supplied in multidose vials but also in single doses.

The following table from the product information sheet describes dosage and delivery forms:

| Strength | Container | Dose (s) | Composition |
|---|---|---|---|
| Spikevax | Multidose | Maximum 10 | One dose (0.5 mL) |
| 0.2 mg/mL | vial (red | doses of | contains 100 |
| dispersion for | flip-off | 0.5 mL each | micrograms of |
| injection | cap) | | elasomeran, a |
| | | | COVID-19 mRNA |
| | | | Vaccine (embedded |
| | | | in lipid |
| | | | nanoparticles). |
| | | Maximum 20 | One dose (0.25 |
| | | doses of | mL) contains 50 |
| | | 0.25 mL each | micrograms of |
| | | | elasomeran, a |
| | | | COVID-19 mRNA |
| | | | Vaccine (embedded |
| | | | in lipid |
| | | | nanoparticles). |
| Spikevax | Multidose | 5 doses | One dose (0.5 mL) |
| 0.1 mg/mL | vial (blue | Of 0.5 mL each | contains 50 |
| dispersion for | flip-off | | micrograms of |
| injection | cap) | | elasomeran, a |
| | | | COVID-19 mRNA |
| | | | Vaccine (embedded |
| | | | in lipid |
| | | | nanoparticles). |
| | Multidose | Maximum 10 | One dose (0.25 |
| | vial (blue | doses of | mL) contains 25 |
| | flip-off | 0.25 mL each | micrograms of |
| | cap) | | elasomeran, a |
| | | | COVID-19 mRNA |
| | | | Vaccine (embedded |
| | | | in lipid |
| | | | nanoparticles). |
| Spikevax | Pre filled | 1 dose of 0.5 mL | One dose (0.5 mL) |
| 50 micrograms | syringe | For single-use | contains 50 |
| dispersion for | | only. | micrograms of |
| injection in | | Do not use the pre- | elasomeran, a |
| pre-filled | | filled syringe to | COVID-19 mRNA |
| syringe | | deliver a partial | Vaccine (embedded |
| | | volume of 0.25 mL. | in lipid |
| | | | nanoparticles). |

Elasomeran is a single-stranded mRNA with a 5′-cap structure embedded in lipid-nanoparticles.

The other ingredients are the following:
SM-102(heptadecan-9-yl 8-{(2-hydroxyethyl) [6-oxo-6-(undecyloxy)hexyl]amino}octanoate)
Cholesterol
1,2-distearoyl-sn-glycero-3-phosphocholine (DSPC)
1,2-Dimyristoyl-rac-glycero-3-methoxypolyethylene glycol-2000 (PEG2000-DMG)
Trometamol
Trometamol hydrochloride
Acetic acid
Sodium acetate trihydrate
Sucrose
Water for injections

US 2003/0147899 A1 discloses a vaccine comprising an endogenous epinephrine and glucocorticoids. The vaccine is said to enhance an antigen specific cell-mediated immune response and protection against an infectious agent, e.g., a virus. Epinephrine and glucocorticoids, such as cortisol, are disclosed as endogenous stress hormones. The combination of those hormones is used as adjuvant or immunomodulator and is said to activate the immune system, while, on the other hand, glucocorticoids themselves are widely used to efficiently suppress immune reactions.

WO 2020/248010 A1 discloses a topical drug delivery formulation to be applied to an open wound and not to be applied subcutaneously. The formulation is intended to avoid rapid peak systemic effects. Among the drugs no vaccine is mentioned. Rather the drug may be a vasopressor as adrenaline (epinephrine). Amongst the delivery formulations no vaccine is disclosed.

US 2004/0062778 A1 refers to a controlled release system intended to target and control the release of an active ingredient. The composition does include a matrix and is no vaccine.

Management of anaphylaxis due to COVID-19 vaccines: In this paper a procedure to treat elderly patient suffering an anaphylactic reaction by intramuscularly administering adrenalin. No disclosure is directed to use adrenalin in a vaccine itself.

It is an object of the invention to provide a vaccine preparation, which upon administration does have no or minor side effects (severe complications).

That object is achieved by a vaccine preparation with the features of claim 1.

Preferred and advantageous embodiments of the vaccine preparation according the invention are subjects of dependent claims.

It was not to be expected that the addition of a vasoconstrictive agent to a vaccine, in particular to a mRNA-vaccine, results in an improved tolerability and less adverse reactions and that this effect can be achieved via a sustained persistence of the vaccine within the muscular tissue.

The invention relies on the assumption, that the distribution of the vaccine within the body determines the amount and type of cells which ultimately take up the vaccine. The addition of a vasoconstrictive agent to the vaccine is expected to result in a sustained persistence within muscular tissue and with a reduced export into surrounding tissues and blood vessels.

In contrast to prior art the combination of a vaccine, in particular a mRNA-vaccine, and a vasoconstrictor, such as adrenalin/epinephrine, does not comprise an adjuvant but rather a therapeutic-drug combination to control the effect of a vaccine, in particular a mRNA based therapeutic. In contrast to conventional vaccines, which contain one or more antigens of a pathogenic microbe as primary ingredient, mRNA therapeutics contain lipid-nanoparticles with a modified synthetic mRNA cargo. Their mode of action differs significantly, as these mRNAs, once taken up by cells, self-amplify and initiate the intracellular production of a protein, e.g. a part of a viral spike-protein. While the idea to use mRNAs as therapeutics was already discussed in the 1990s [Gaviria, M. & Kilic, B. A network analysis of COVID-19 mRNA vaccine patents. Nat. Biotechnol. 39, 546-548 (2021) and Wolff, J. A. et al. Direct Gene Transfer into Mouse Muscle in Vivo, Science 247, 1465-1468 (1990)], the basic technology to make this even possible was not developed before 2005 [Gaviria, M. & Kilic, B. A network analysis of COVID-19 mRNA vaccine patents. Nat. Biotechnol. 39, 546-548 (2021) and Karikó, K. Buckstein, M., Ni, H. & Weissmann, D. Suppression of RNA Recognition by Toll-like Receptors: The Impact of Nucleoside Modification and the Evolutionary Origin of RNA. Immunity 23, 165-175 (2005)], which also was long before the first mRNA based vaccines were developed by Pfizer-Biontech and Moderna [Baden, L. R. et al. Efficacy and Safety of the mRNA-1273 SARS-CoV-2 Vaccine. N. Engl. J. Med. 384, 403-416 (2021) and Benjamini, O. et al. Safety and efficacy of the BNT162b mRNA COVID-19 vaccine in patients with chronic lymphocytic leukemia. Haematologica 107, 625-634 (2021) and Chaudhary, N., Weissmann, D. & Whitehead, K. A. mRNA vaccines for infectious diseases: principles, delivery and clinical translation. Nat. Rev. Drug Discov. 20, 817-838 (2021)].

In conclusion the adjuvant for conventional vaccines disclosed in US 2003/0147899 A1 could not have been intended for the use with mRNA-vaccines, since these do not only comprise a completely different technology, but also did not yet exist at that time.

The invention is essentially suitable for all vaccines which are applied via intramuscular injection, but is especially beneficial for mRNA-vaccines.

The vasoconstrictive agents, which were considered for the application in the context of the invention, are adrenalin/epinephrine, angiotensin I and II, serotonin, thromboxane A2, endothelin, phenylephrine, α-methylnorepinephrine, felypressin and noradrenalin/norepinephrine. Also, combinations of at least two of the aforementioned agents are taken into consideration.

### EXAMPLES

The practical examples are formulated in accordance to the containers which are supplied by the manufacturers.

### Example 1

Combination of epinephrine with Comirnaty (0.45 mL) vial for 6 doses. The combination contains 0.01 mg of epinephrine per dose (0.3 mL after dilution).
0.45 ml Comirnaty (stock)
0.06 mg epinephrine

### Example 2

Combination of levonordefrin with Comirnaty (0.45 mL) vial for 6 doses. The combination contains 0.05 mg of levonordefrin per dose (0.3 mL after dilution).
0.45 ml Comirnaty (stock)
0.3 mg levonordefrin

### Example 3

Combination of norepinephrine with Comirnaty (0.45 mL) vial for 6 doses. The combination contains 0.033 mg of norepinephrine per dose (0.3 mL after dilution).
0.45 ml Comirnaty (stock)
0.2 mg norepinephrine

### Example 4

Combination of epinephrine with Spikevax dispersion in an injection ready syringe. The combination contains a single dose (50 micrograms) of 0.5 mL Spikevax dispersion with an addition of 0.01 mg epinephrine.
0.5 mL Spikevax (dispersion)
0.01 mg epinephrine

### Example 5

Combination of levonordefrin with Spikevax dispersion in an injection ready syringe. The combination contains a single dose (50 micrograms) of 0.5 mL Spikevax dispersion with an addition of 0.05 mg levonordefrin.
0.5 mL Spikevax (dispersion)
0.05 mg levonordefrin

### Example 6

Combination of norepinephrine with Spikevax dispersion in an injection ready syringe. The combination contains a single dose (50 micrograms) of 0.5 mL Spikevax dispersion with an addition of 0.033 mg norepinephrine.
0.5 mL Spikevax (dispersion)
0.033 mg norepinephrine

In conclusion a practical example of the invention may be described as follows:

A vaccine preparation with no or little side adverse effects and an improved tolerability of vaccines, such as mRNA-vaccines, additionally contains at least one vasoconstrictive agent, such as adrenalin.

1. Vaccine preparation, comprising at least one vaccine, wherein the vaccine preparation comprises in addition to the vaccine at least one vasoconstrictive agent.

2. Preparation according to claim 1, wherein the vaccine is a mRNA-vaccine.

3. Preparation according to claim 2, wherein the vaccine is a mRNA vaccine against COVID-19.

4. Preparation according to claim 1, wherein the vasoconstrictive agent is at least one selected from the group comprising adrenaline/epinephrine, norepinephrine, angiotensine I and II, serotonine, thromboxane A2, endotheline, phenylephrine, levonordephrine and alpha-noradrenaline/norephinephrine.

5. Preparation according to claim 1, wherein the vaccine preparation is made for intramuscular administration.

6. Preparation according to claim 1, wherein the vaccine preparation contains in addition to the vaccine from 0,005 to 0,1 mg, in particular from 0,01 to 0,05 mg, vasoconstrictive agent per dose of vaccine preparation to be administered.
